(19) United States
(12) Patent Application Publication
(10) Pub. No.: US 2010/0095591 A1
(43) Pub. Date: Apr. 22, 2010

(54) EMISSIONS CONTROL SYSTEM AND METHOD

(75) Inventors: Dan Hancu, Clifton Park, NY (US); Ashish Balkrishna Mhadeshwar, Schenectady, NY (US); Daniel George Norton, Niskayuna, NY (US); Frederic Vitse, Knoxville, TN (US); Benjamin Hale Winkler, Albany, NY (US)

Correspondence Address: GENERAL ELECTRIC COMPANY;GLOBAL RESEARCH, ONE RESEARCH CIRCLE, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 12/254,224

(22) Filed: Oct. 20, 2008

(51) Int. Cl. C10J 3/00; B01J 19/00; C07C 7/148

(52) U.S. Cl. 48/211; 422/188; 585/832

(57) ABSTRACT

A system includes a fuel converter comprising a catalyst composition, and the catalyst composition can convert fuel into a hydrocarbon reductant stream; a separation system that separates the hydrocarbon reductant stream into a first reductant sub-stream that comprises short chain hydrocarbon molecules, and a second reductant sub-stream that comprises long chain hydrocarbon molecules; a selective catalytic reduction catalyst reactor in fluid communication with the fuel converter, and the catalyst reactor has an inner surface that defines a first zone and a second zone, and the first zone is configured to receive the second reductant sub-stream, and the second zone is configured to receive the first reductant sub-stream; and an exhaust stream that flows into the first zone contacts the second reductant sub-stream before flowing into the second zone and contacting the first reductant sub-stream.

### BACKGROUND OF THE INVENTION

The present disclosure includes embodiments that relate to systems for controlling emissions. The disclosure further includes embodiments that relate to a method for controlling emissions.

Some vehicles may emit nitrogen oxides (NOx) during use. Such emissions may be undesirable.

Emission controls have included engine modification and exhaust gas treatment. It may be desirable to have a system for emissions control that differs from those systems currently available. It may be desirable to have a method of controlling emissions that differs from those methods that currently available.

### BRIEF DISCUSSION OF THE INVENTION

Disclosed herein are systems and methods for controlling emissions. In one embodiment, the method of controlling emissions includes a system, comprising a fuel converter comprising a catalyst composition, and the catalyst composition can convert fuel into a hydrocarbon reductant stream; a separation system that separates the hydrocarbon reductant stream into a first reductant sub-stream that comprises short chain hydrocarbon molecules, and a second reductant sub-stream that comprises long chain hydrocarbon molecules; a selective catalytic reduction catalyst reactor in fluid communication with the fuel converter, and the catalyst reactor has an inner surface that defines a first zone and a second zone, and the first zone is configured to receive the second reductant sub-stream, and the second zone is configured to receive the first reductant sub-stream; and an exhaust stream that flows into the first zone contacts the second reductant sub-stream before flowing into the second zone and contacting the first reductant sub-stream.

Another embodiment includes a method comprising converting a fuel into a hydrocarbon reductant stream; separating the hydrocarbon reductant stream into a plurality of sub-streams, and each of the plurality of streams has a hydrocarbon reductant with a differing average carbon chain length; feeding the plurality of streams to a selective catalytic reduction catalyst reactor, wherein each of the plurality of sub-streams is fed to a corresponding zone in the reactor so as to contact one of a set of catalyst compositions, and each catalyst composition in the set being configured to function in a determined manner with the carbon chain length of the hydrocarbon reductant of that sub-stream; and contacting an exhaust stream with the selective catalytic reduction catalyst reactor and the plurality of hydrocarbon reductant sub-streams to control a concentration of one or more components of the exhaust stream.

The above described and other features are exemplified by the following Figures and detailed description.

### BRIEF DESCRIPTION OF THE DRAWINGS

Referring to the exemplary drawings wherein like elements are numbered alike in the several Figures:

FIG. 1 is a schematic illustration of an embodiment of a system for controlling emissions;

FIG. 2 is a schematic illustration of another embodiment of a system for controlling emissions;

FIG. 3 is a schematic illustration of still another embodiment of a system for controlling emissions;

FIG. 4 is a schematic illustration of still another embodiment of a system for controlling emissions; and

FIG. 5 is a schematic illustration of still another embodiment of a system for controlling emissions.

### DETAILED DESCRIPTION OF THE INVENTION

The present disclosure includes embodiments that relate to systems and methods of controlling emissions. Systems and methods for controlling emissions may reduce the nitrogen oxides (NOx) emissions from the exhaust stream of a vehicle or a stationary source. Vehicles may include, for example, locomotives, marine vessels, off-highway vehicles, tractor-trailer rigs, passenger vehicles, and the like. Emissions control refers to the ability to affect the compositional make up of an exhaust gas stream. As exhaust gas is a mixture of components, the reduction of one component almost invariably increases the presence of another component. For clarity of discussion, the chemical reduction of NOx is used as a non-limiting example of emission reduction insofar as the concentration of a determined species within the exhaust gas stream is controlled.

The system utilizes the fuel for the engine as a reductant to reduce NOx emissions. The system converts the already on-board fuel into a broad range or reductants that can be categorized by the length of their hydrocarbon chains. The categories are mentioned in reference to the number of carbon atoms they comprise, for example, chains having one to fourteen or more carbon atoms are referred to as C1 to C14 reductants. These reductants display a broad range of reducing power with the longer hydrocarbon chains (i.e., C5-C10) reducing NOx at lower temperatures, and shorter chains (i.e., C1-C4), reducing NOx at higher temperatures. The fuel reductants are mixed with the exhaust stream and facilitate a reduction of NOx emissions in the presence of a hydrocarbon based selective catalytic reduction (SCR) catalyst reactor. The NOx emissions are thereby reduced from the vehicle emissions. The system can be utilized on board in all types of vehicles that employ internal combustion or compression engines powered by hydrocarbon-based fossil fuels. The system can also be utilized on board in all types of locomotives that employ engines and turbines powered by hydrocarbon-based fossil fuels. In particular, the system can be utilized in vehicles that employ diesel engines. Advantageously, the system described herein does not require the need for additional reductant chemicals or the storage equipment required to be on-board therewith.

As used herein, the term “exhaust stream” refers to a composition comprising NOx produced by a combustion process. The exhaust stream further may comprise carbon monoxide (CO), carbon dioxide (CO2), molecular nitrogen (N2), molecular oxygen (O2), which can serve as a combustion fuel for the hydrocarbon reductant at increased temperatures, or incompletely combusted fuel may also be present in the exhaust stream. Also as used herein, the fuel described as being converted into the various reductants means a fuel being combusted by the engine of the vehicle, locomotive, generator, or the like. Exemplary primary fuels include, without limitation, diesel, gasoline, jet-fuel, fuel oil, bio-fuels, such as bio-diesel, and the like, or a combination comprising at least one of the foregoing hydrocarbon-based fuels. Also, in the following description, an “upstream” direction refers to the direction from which the local flow is coming, while a “downstream” direction refers to the direction in which the local flow is traveling. In the most general sense, flow through the system tends to be from front to back so the “upstream direction” will generally refer to a forward direction, while a “downstream direction” will refer to a rearward direction. The terms reducing agent and reductant are used interchangeably throughout this disclosure. The term “fluid communication” is intended to encompass the containment and/or transfer of compressible and/or incompressible fluids between two or more points in the system. Examples of suitable fluids are gases, liquids, combinations of gases and liquids, or the like. The use of pressure transducers, thermocouples, injectors, flow, hydrocarbon, and NOx sensors aid in communication and control. In one embodiment, computers can be used to aid in the flow of fluids in the system. The term “on-board” refers to the ability of a vehicle or locomotive to host the system in its entirety aboard the vehicle or locomotive.

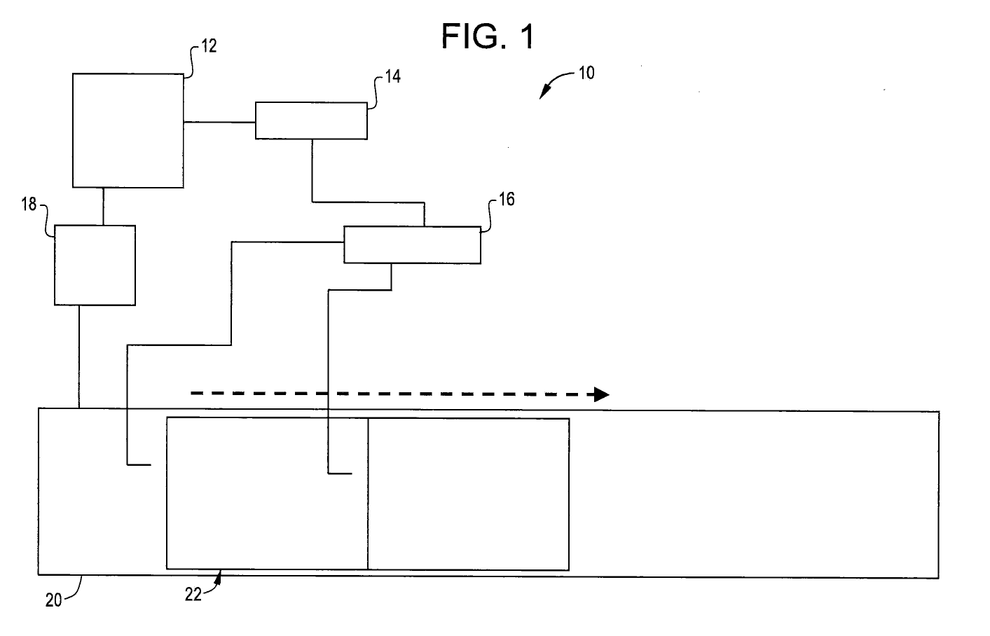

Referring now to FIG. 1, an exemplary embodiment of the system 10 for reducing nitrogen oxides emissions is illustrated. Advantageously, the system 10 can be employed in both stationary applications as well as mobile applications such as vehicle systems (e.g., locomotives, trucks, and the like). The system 10 comprises a fuel tank 12, a fuel converter 14, a separation system 16, an engine 18, and an exhaust conduit 20. The exhaust conduit 20 comprises the SCR catalyst reactor 22, through which the exhaust stream flows. The fuel tank 12 is upstream of the fuel converter 14 and the separation system 16. The fuel tank 12, the fuel converter 14, the separation system, and the exhaust conduit 18 are in fluid communication with one another. The fuel converter 14 is located between the fuel tank 12 and the separation system 16. The engine 18 is located downstream of the fuel tank 12 and in fluid communication with the fuel tank 12. The engine 18 is located upstream of in fluid communication with the exhaust conduit 20.

In general, the fuel converter 14 converts the engine fuel into a range of reductants of determined hydrocarbon chain length. The reductants can proceed to the separation system 16, where the reductants can be separated into one or more streams based on the hydrocarbon chain length. Depending on the length of the hydrocarbon chains in the reductants, the different streams can be sent to varying locations of the exhaust conduit 20 based on the temperature of the location in the conduit and the particular catalyst bed of the SCR catalyst reactor 22. For example, diesel can be processed on-board in the fuel converter 14 through various methods including autothermal cracking, catalytic partial oxidation (CPO), and the like, to produce hydrogen and hydrocarbon reductants usually ranging from C1 to C14. As stated above, the reducing power of these hydrocarbons is dependent on the length of the hydrocarbon chain. The system 10 allows for a flexible SCR catalytic process using multiple optimized catalytic beds to provide optimum utilization of the broad diversity of hydrocarbon reductants produced in the fuel converter 14 and separated by the separation system 16. By combining the proper set of SCR catalysts in the proper order (from upstream to downstream), and by injecting the proper portion of hydrocarbon-based reductants at proper locations in the SCR catalyst process, NOx conversion can be optimized.

A variety of fuels may be stored in the fuel tank 12 and used in the system 10. The primary fuel tank supplies fuel to the engine 18. As mentioned above, the engine 18 can be any spark ignition engine, or compression ignition engine. While spark ignition engines are referred to as gasoline engines and compression ignition engines are referred to as diesel engines, it is to be understood that various other types of hydrocarbon based fuels can be employed in the respective internal combustion engines. As mentioned, in an exemplary embodiment, the primary hydrocarbon-based fossil fuel is a liquid fuel. As will be discussed in greater detail below, the fuel converter 14 converts the fuel to C1-C14 hydrocarbon reductants and/or hydrogen and carbon monoxide, which can then be used to reduce NOx in the exhaust stream depending on the exhaust temperature. Long chain hydrocarbons are hydrocarbons that have 9 or more carbon atoms. In an exemplary embodiment, an exemplary long chain hydrocarbon primary fuel is diesel.

As shown, the exhaust stream from the engine 18 is disposed into the exhaust stream conduit 22, which contains the SCR catalyst reactor 22. The SCR catalyst reactor 22 comprises a plurality of selective catalyst reduction beds optimized for a HC-SCR process. In this embodiment, the reactor is shown having two beds to receive the two separated hydrocarbon reductant streams from the separation systems 16. The reduction beds each comprise a catalyst suitable for the reductant being fed thereover, which is typically placed at a location within the exhaust conduit where it will be exposed to the exhaust stream containing the NOx. The catalyst may be arranged as a packed or fluidized bed reactor, coated on a monolithic or membrane structure, or arranged in any other manner within the exhaust system such that the catalyst is in contact with the effluent gas.

The fuel converter 14 is a fixed bed reactor that is configured to perform an autothermal cracking and/or a catalytic partial oxidation processes to form the hydrocarbon and/or the hydrogen reductants respectively. A gas-assisted nozzle can be utilized to atomize the fuel at a low-pressure inlet into the fuel converter. The atomized fuel can then be converted via one of the processes into a hydrocarbon reductant (e.g., C1-C14) or a mixture of hydrogen and carbon monoxide. Partial oxidation is relatively selective toward carbon monoxide and hydrogen gas productions and these compounds are particularly effective at reducing NOx at exhaust stream temperatures of about 375 degrees Celsius or lower. Autothermal cracking provides the broad range of hydrocarbon reductants. The range of reductants display an equally broad range of reducing power as stated above.

The fixed bed reactor of fuel converter 14 comprises a catalyst composition. In an exemplary embodiment, the catalyst composition is able to operate under conditions that vary from oxidizing at the inlet of the converter to reducing conditions at the exit of the converter. The catalyst can be capable of operating effectively and without any thermal degradation from a temperature in a range of from about 200 degrees Celsius to about 900 degrees Celsius. The catalyst can operate effectively in the presence of air, carbon monoxide, carbon dioxide, water, alkanes, alkenes, cyclic and linear compounds, aromatic hydrocarbons and sulfur-containing compounds. The catalyst composition can provide for low levels of coking such as by preferentially catalyzing the reaction of carbon with water to form carbon monoxide and hydrogen thereby permitting the formation of only a low level of carbon on the surface of the catalyst. Moreover, an exemplary catalyst composition may satisfy all of the foregoing requirements simultaneously.

The catalyst composition of the fuel converter 14 is bifunctional, i.e., it performs the autothermal cracking function and the catalytic partial oxidation function. The cracking function involves the breaking of the hydrocarbon-based fossil fuel molecules (e.g., diesel) into shorter molecules to extract low-boiling fractions of varying hydrocarbon chain lengths. An exemplary cracking function involves the breaking of heavy hydrocarbon molecules found in diesel fuel to light hydrocarbon reductant molecules having backbone chains of fourteen or less carbon atoms.

The catalytic partial oxidation function involves the oxidation of the fuel hydrocarbons into carbon monoxide and hydrogen. The catalyst composition can generally comprise sites that perform the catalytic partial oxidation function (catalytic partial oxidation sites) located adjacent to sites that perform the cracking function (cracking sites).

In one embodiment, the catalyst composition contained in the fuel converter 14 is bifunctional, i.e., it serves to crack longer chain hydrocarbons of the fuel to a broad range of hydrocarbon reductants having one to about fourteen carbon atoms. The bifunctional catalyst slows down coke build-up rate on the surface of cracking catalysts, thus allowing it to continue being active for cracking hydrocarbons, which would normally not occur on conventional cracking catalysts operating under similar conditions. In the catalyst composition, since the catalytic partial oxidation reaction is an exothermic reaction, while cracking is an endothermic reaction, the heat generated at a catalytic partial oxidation site facilitates the endothermic cracking reaction and also facilitates the oxidation of coke. In one embodiment, the catalytic partial oxidation sites are used to oxidize the coke away from the cracking sites to keep the cracking sites clean and active.

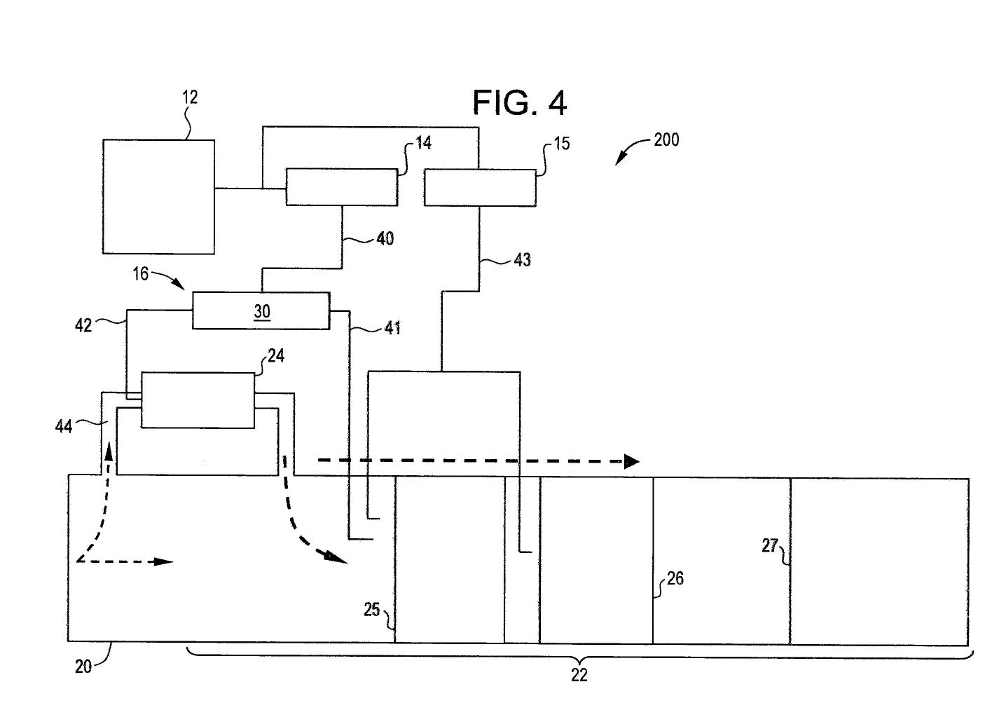

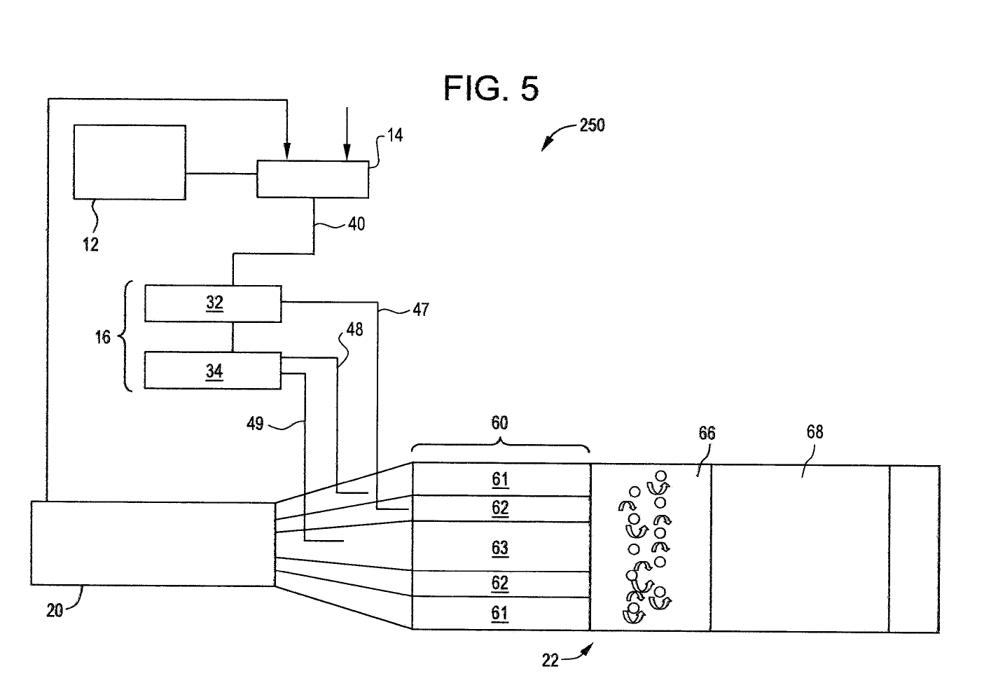

The use of a fuel converter 14 that employs the catalytic composition is advantageous in that it may use only a single fixed bed reactor to convert diesel fuel to a mixture of hydrocarbon reductants and hydrogen gas. If desired, the fuel converter 14 can employ more than one fixed bed reactor to improve productivity. For example, the catalytic converter can employ about 2 to about 6 fixed bed reactors if desired. As shown in FIGS. 1-3, the autothermal cracking and catalytic partial oxidation processes take place in a single fuel converter unit. In another embodiment, as shown in FIGS. 4-5, one fuel converter 14 can be used to produce the hydrocarbon reductants from the fuel, while a second fuel converter 15 can comprise a CPO reformer for producing the hydrogen gas reductant along with carbon monoxide.

The catalytic partial oxidation sites generally comprise noble metals that perform the catalytic partial oxidation function. The catalytic partial oxidation sites comprise one or more “platinum group” metal components. As used herein, the term “platinum group” metal implies the use of platinum, palladium, rhodium, iridium, osmium, ruthenium or mixtures thereof Exemplary platinum group metal components are rhodium, platinum and optionally, iridium. The catalyst composition includes an amount of material in a range of from about 0.1 wt % to about 20 wt % of the platinum group metal. The platinum group metal components optionally may be supplemented with one or more base metals. In one embodiment, the base metal may be of Group III, Group IB, Group VB and Group VIB of the Periodic Table of Elements. Exemplary base metals are iron, cobalt, nickel, copper, vanadium and chromium.

The cracking sites may include a zeolite. The zeolites may have a silica-to-alumina mole ratio of at least about 12. In one embodiment, a zeolite having a silica-to-alumina mole ratio of about 12 to about 1000 is used. In one embodiment, a zeolite having a silica-to-alumina mole ratio of about 15 to about 500 is used. Examples of suitable zeolites include RE-Y (rare earth substituted yttria), USY (ultrastable yttria zeolite), RE-USY ZSM-5, ZSM-11, ZSM-12, ZSM-35, zeolite beta, MCM-22, MCM-36, MCM-41, MCM-48, or the like. Also suitable are combinations that include at least one of the foregoing zeolites.

Zeolites also contemplated for use in this process are the crystalline silicoaluminophosphates (SAPO). Examples of suitable silicoalumino-phosphates include SAPO-11, SAPO-34, SAPO-31, SAPO-5, SAPO-18, or the like, or a combination comprising at least one of the foregoing silicoaluminophosphates.

The platinum group catalysts along with other base metal catalysts are washcoated onto the molecular sieves to form the catalytic composition. The catalytic partial oxidation sites comprise about 0.1 to about 5.0 weight percent (wt %) of the total weight of the catalytic composition. In one embodiment, the catalytic partial oxidation sites may include an amount about 0.3 to about 1.0 wt % of the total weight of the catalytic composition.

A portion of the hot exhaust gas that is emitted by the engine can be used as a secondary gas for atomizing the primary fuel in the fuel converter 14. Air can also be employed as the secondary gas for atomizing the primary fuel. In an exemplary embodiment, a portion of the exhaust stream is combined with air to form the secondary gas to facilitate the catalytic partial oxidation reaction. The amount of hot engine exhaust gas is effective to light off the catalytic partial oxidation reaction in the fuel converter 14. Water present in the exhaust stream can facilitate further the reduction of coke formation on the catalyst.

In one embodiment, the hydrocarbon reductants and the hydrogen gas reductant leaving the fuel converter 14 all flow to the separation system 16. In another embodiment, such as that shown in FIGS. 4 and 5, the hydrogen gas reductant (along with the carbon monoxide) are fed directly to the SCR catalyst reactor 22 in the exhaust conduit 20. Regardless of the embodiment, at least the hydrocarbon reductants are fed to the separation system 16 for separation into one or more reductant streams based on the number of carbon atoms in the hydrocarbon chain.

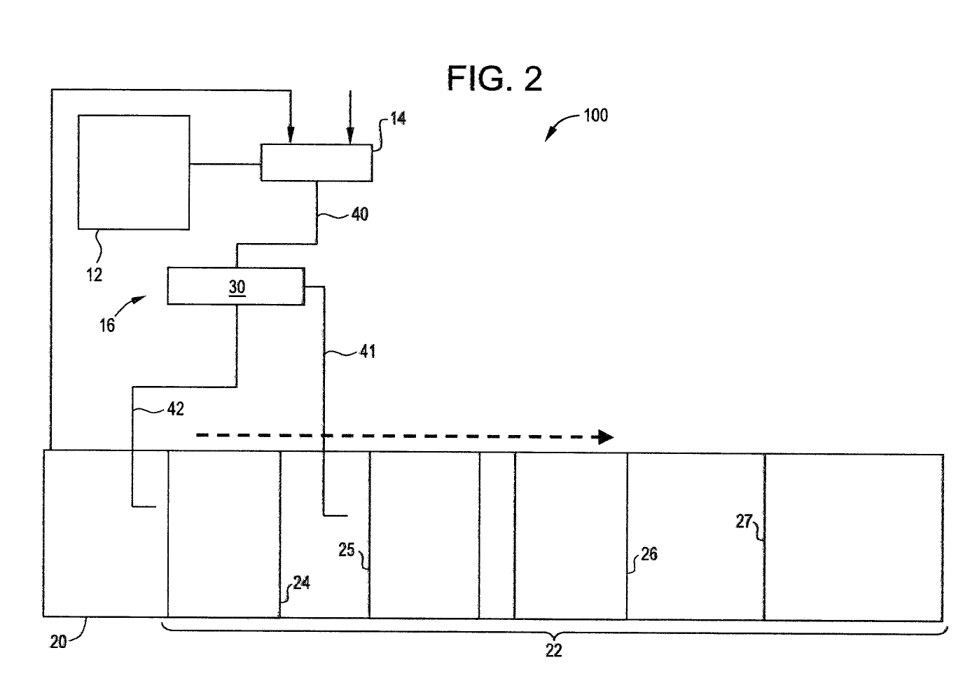

The system 10, therefore, employs a separation system 16. The separation system 16 is generally configured to divide the hydrocarbon reduction stream from the fuel converter 14 into at least two streams to be fed to the exhaust conduit 20. Separating the lower carbon atom containing reductant from the higher carbon atom containing reductant separates or divides the hydrocarbon reductant stream. The separation between the hydrocarbon reductant streams can be achieved in the system 16 based on the difference in volatility observed for the different lengths of carbon chains. The separator system 16 can comprise separators such as distillation columns (with optional vacuum systems), packed columns, membranes, condensers, centrifuges, or the like that can be used to separate C11 and higher hydrocarbons from C1-10 hydrocarbons. For example, a set of condensers and distillation columns can be ordered with specific temperature profiles tuned to achieve the proper separation for a given hydrocarbon chain length. In one embodiment, the NOx reducing system can include only a single separator. In another exemplary embodiment, the NOx reducing system can include a separation system having two or more separators (as shown in FIG. 2). In one embodiment, the system can include separators that can separate one set of hydrocarbon reductants (e.g., long chain hydrocarbons) from another set of hydrocarbon reductants (e.g., short chain hydrocarbons). Similarly, C1-C4 hydrocarbon reductants can be separated from the C5-C10 hydrocarbon reductants, and so on. Short chain hydrocarbons, as used herein, include those organic molecules having up to about 10 carbon per molecule, with long chain hydrocarbons having more. Due to branching, heteroatoms, and the like the molecular weight and exact carbon count may be selected based on end use parameters.

As can be seen in FIGS. 1-5, the separation system 16 is located down stream of the fuel converter 14 and upstream of the SCR catalyst located in the SCR catalyst reactor 22. The separation system 16 is in fluid communication with both the fuel converter 14 and the SCR catalyst reactor 22. In one exemplary embodiment, the separation system 16 includes a condenser 30. In another exemplary embodiment, the separation system includes a first condenser 32 and a second condenser 34. Referring to FIG. 2, the NOx reducing system 100 includes a condenser 30 disposed in fluid communication with the fuel converter 14 for condensing at least a portion of the hydrocarbon and hydrogen gas combined reductant stream 40 exiting the converter. Accordingly, the hydrocarbon reductant in the combined stream 40 can be condensed in the condenser 30 and injected into the exhaust stream as a liquid, or it can be condensed and then stored in a holding tank (not shown). The condensed stream 41 includes C1-C10 hydrocarbon reductant and the hydrogen reductant. Upon exiting the separation systems 16, the condensed stream 41 can be fed to the SCR catalyst reactor 22. The heavy hydrocarbon stream 42 includes C11 and above hydrocarbon reductants and is also fed to the SCR catalyst reactor 16 for treating the exhaust stream. In the embodiment of FIG. 2, the heavy hydrocarbon stream 42 is fed to a first zone 24 of the SCR catalyst reactor 22. The first zone 24 can include a deep oxidation catalyst (DOC) bed comprising a catalytic metal. The DOC bed of the first zone 24 can be configured to catalytically combust the heavy hydrocarbon stream 42. The combustion of the heavy hydrocarbons can increase the exhaust stream temperature to further aid in the NOx reduction of the downstream zones. The DOC can also simultaneously partially convert nitrogen oxide to nitrogen dioxide as the heavy hydrocarbons are combusted. In one embodiment, the catalytic metal of the DOC bed includes platinum, palladium, or a mixture thereof The DOC can be prepared by techniques well known to those skilled in the art. Alternatively, the DOC can be obtained from commercial sources.

The condensed stream 41 is fed to the second and third zones 25 and 26 of the SCR catalyst reactor 22. The second zone 25 can include a catalyst bed configured to react the C5-C10 hydrocarbons with the NO in the exhaust stream. The remaining C1-C4 hydrocarbons can travel to the third zone 26 unreacted, because the exhaust temperature in the second zone 25 is not yet high enough for the C1-C4 hydrocarbons to convert the NOx. The catalyst bed of the second zone 25 can have any catalyst composition configured to react the C5-C10 hydrocarbons with the exhaust stream. In one embodiment, the catalyst composition includes a low silver (Ag) content. Exemplary Ag contents can be about 0.5 percent by weight (wt %) to about 10 wt %, specifically about 1 wt % to about 6 wt %, more specifically about 2 wt %, based on the total weight of the catalyst composition. In an exemplary embodiment, the Ag content can be deposited on an aluminum oxide support (Al₂O₃) to form the catalyst bed of the second zone.

Likewise, the third zone can include a catalyst composition of higher Ag content configured for reacting the lower (C1-C4) hydrocarbons with the exhaust stream at a temperature higher than the first or second zones of the SCR catalyst reactor. Exemplary Ag contents for the third zone catalyst composition can be about 1 wt % to about 10 wt %, specifically about 2 wt % to about 8 wt %, more specifically about 6 wt %. In an exemplary embodiment, the Ag content can be deposited on an aluminum oxide support (Al₂O₃) to form the catalyst bed of the third zone.

In general, SCR catalysts are those catalyst materials that enable the chemical reduction of NOₓ species to less harmful constituents such as diatomic nitrogen (i.e., N₂). Many of the SCR catalyst materials that promote reduction of NOₓ species via reaction with an exhaust stream and reductants may be suitable for use in embodiments of the system described herein. For example, silver on an Alumina support that is coated on a monolith support structure may be used. In particular, 3.0% silver on mesoporous alumina that is coated on a monolith core has been found to be particularly effective in embodiments described herein. In another embodiment, the SCR catalyst compositions comprise zeolites.

In still another embodiment, the SCR catalyst composition can comprise a catalytic metal disposed upon a substrate that has pores of a size effective to prohibit aromatic species from poisoning the catalyst composition. The pores generally have an average pore size of about 2 to about 50 nanometers when measured using nitrogen measurements. The catalytic metal comprises alkali metals, alkaline earth metals, transition metals, and main group metals. Examples of suitable catalytic metals are silver, platinum, gold, palladium, iron, nickel, cobalt, gallium, indium, ruthenium, rhodium, osmium, iridium, or the like, or a combination comprising at least one of the foregoing metals.

The average catalytic metal particle size is about 0.1 to about 50 nanometers. The catalytic metals are present in the catalyst composition in an amount of about 0.025 to about 50 mole percent (mol %). In one embodiment, the catalytic metals are present in the catalyst composition in an amount of about 1 to about 40 mol %. In one embodiment, the catalytic metals are present in the catalyst composition in an amount of about 1.5 to about 35 mol %. An exemplary amount of catalytic metal in the catalyst composition is about 1.5 to about 5 mol %.

The substrate for the catalyst can generally be meso-porous and comprises an inorganic material such as for example, a metal oxide, inorganic oxides, inorganic carbides, inorganic nitrides, inorganic hydroxides, inorganic oxides having hydroxide coatings, inorganic carbonitrides, inorganic oxynitrides, inorganic borides, inorganic borocarbides, or the like, or a combination comprising at least one of the foregoing inorganic materials. Examples of suitable inorganic materials are metal oxides, metal carbides, metal nitrides, metal hydroxides, metal oxides having hydroxide coatings, metal carbonitrides, metal oxynitrides, metal borides, metal borocarbides, or the like, or a combination comprising at least one of the foregoing inorganic materials. Metallic cations used in the foregoing inorganic materials can be transition metals, alkali metals, alkaline earth metals, rare earth metals, or the like, or a combination comprising at least one of the foregoing metals.

Examples of suitable inorganic oxides include silica (SiO2), alumina (Al2O3), titania (TiO2), zirconia (ZrO2), ceria (CeO2), manganese oxide (MnO2), zinc oxide (ZnO), iron oxides (e.g., FeO, β-Fe2O3, γ-Fe2O3, ε-Fe2O3, Fe3O4, or the like), calcium oxide (CaO), manganese dioxide (MnO2 and Mn3O4), or combinations comprising at least one of the foregoing inorganic oxides. Examples of inorganic carbides include silicon carbide (SiC), titanium carbide (TiC), tantalum carbide (TaC), tungsten carbide (WC), hafnium carbide (HfC), or the like, or a combination comprising at least one of the foregoing carbides. Examples of suitable nitrides include silicon nitrides (Si3N4), titanium nitride (TiN), or the like, or a combination comprising at least one of the foregoing. Examples of suitable borides are lanthanum boride (LaB6), chromium borides (CrB and CrB2), molybdenum borides (MoB2, Mo2B5 and MoB), tungsten boride (W2B5), or the like, or combinations comprising at least one of the foregoing borides. An exemplary inorganic substrates is mesoporous alumina. The mesoporous alumina may be crystalline or amorphous.

The average pore size of the mesoporous substrate obviates poisoning by aromatic species present in the reductant or in the exhaust stream. It is therefore desirable for the substrate to have average pores sizes of about 2 nanometers to about 50 nanometers. In one embodiment, the substrate can have average pores sizes of about 3 to about 20 nanometers. In another embodiment, the substrate can have average pores sizes of about 4 to about 10 nanometers.

The pores can have a narrow distribution in pore sizes. In one embodiment, the pores can have a pore size distribution polydispersity index that is less than about 1.5. In one embodiment, the pores can have a pore size distribution polydispersity index that is less than about 1.3. In another embodiment, the pores can have a pore size distribution polydispersity index that is less than about 1.1. In an exemplary embodiment, the distribution in diameter sizes can be monodisperse. The mesoporous materials can be manufactured via a templating process, which will be described below.

In an exemplary embodiment, the pores are ordered. In one embodiment, the pores are unidirectional and have an average periodicity. In another embodiment, the pores are randomly distributed.

The porous substrate generally has a surface area of about 100 to about 2,000 m²/gm. In one embodiment, the porous substrate has a surface area of about 200 to about 1,000 m²/gm. In another embodiment, the porous substrate has a surface area of about 250 to about 700 m²/gm.

The porous substrate is generally present in the catalyst composition in an amount of about 50 to about 99.9975 mol %, of the catalyst composition. In one embodiment, the porous substrate is generally present in the catalyst composition in an amount of about 60 to about 99 mol %, of the catalyst composition. In another embodiment, the porous substrate is generally present in the catalyst composition in an amount of about 65 to about 98.5 mol %, of the catalyst composition. An exemplary amount of porous substrate in the catalyst composition is about 95 to about 98.5 mol %, of the catalyst composition.

Finally, a fourth zone 27 in the SCR catalyst reactor 22 can be included. The fourth zone 27 can be configured to react any remaining unconverted NOx with by-products of the NOx reduction achieved in the previous zones. By products of the reduction are generally nitrogen-containing compounds sometimes referred to as RONO. The catalyst composition in the fourth zone 27 can include a RONO destruct catalyst (RDC) configured to convert at least a portion of the remaining NOx and RONO into nitrogen. The RDC composition can include a catalytic metal, wherein the catalytic metal includes indium, copper, manganese, tungsten, molybdenum, titanium, vanadium, iron, cerium, or mixtures thereof.

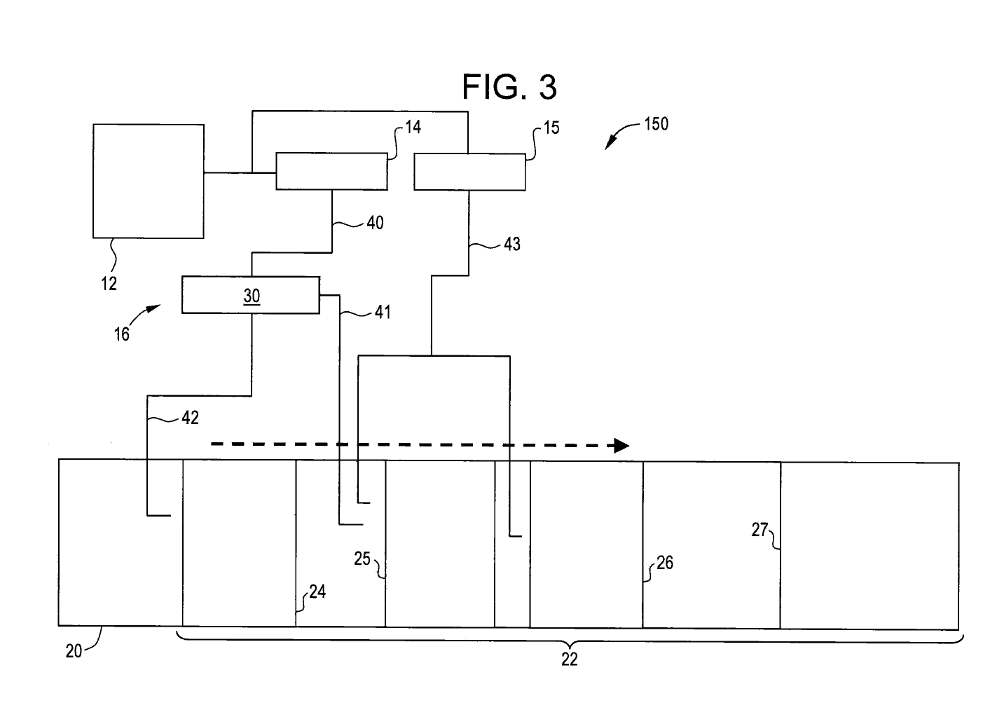

Referring now to FIG. 3, the condenser 30 feeds the two streams from the separation system 16 to the exhaust stream in the same manner as the system of FIG. 2. In this NOx reducing system 150, however, the fuel converter 14 includes a separate additional catalytic partial oxidation reformer 15 that separately feeds the hydrogen reductant and the carbon monoxide stream 43 that has been converted from the fuel directly to the second and third zones of the SCR catalyst reactor 22, bypassing the separation system 16 altogether. In this particular embodiment of the system, the hydrogen acts as a co-reductant with the hydrocarbon reductants to aid in the reduction of the NOx emissions. The hydrogen can be particularly effective at lower temperatures, such as below about 375° C., for example.

The NOx reducing system 200 of FIG. 4 is similar to the system 150 of FIG. 3. The main difference in the embodiment of FIG. 4 is that the first zone 24 of the SCR catalyst reactor 22 is removed from the exhaust conduit 20. In this embodiment, a partial exhaust stream 44 is diverted from the main stream flowing through the conduit and is fed to the DOC bed of the first zone 24. The C11 and above heavy hydrocarbons from the condenser 30 are fed to the first zone 24 and are combusted while simultaneously partially converting the NOx in the partial exhaust stream 44. The hotter, partially reduced, exhaust stream is then fed back into the exhaust conduit 20 where it is fed to the remaining zones of the SCR catalyst reactor 22.

Referring now to FIG. 5, an exemplary embodiment of a NOx reducing system 250 is illustrated. In this system, the separation system 16 includes two condensers 32 and 34. The converted hydrocarbon reductant stream 40 passes through the first condenser 32 where the light hydrocarbons (C1-C4) and the hydrogen reductant are condensed out into the first reductant feedstream 47. The remaining contents of the converted hydrocarbon reductant stream 40 are then sent to the second condenser 34. The second condenser 34 is configured to separate the remaining hydrocarbons into two additional feedstreams. The second reductant feedstream 48 includes C5-C10 hydrocarbons. The third reductant feedstream 49 includes the remaining C11 and above hydrocarbons. In this particular embodiment, rather than having successive catalyst bed zones disposed in an upstream-to-downstream fashion, the SCR catalyst reactor of the system 250 includes a multi-tiered first zone 60. Each of the reductant feedstreams is fed to a different tier of the first zone 60. A predetermined temperature for reacting the reductant with the catalysts can depend on the final catalyst composition in the zones, exhaust temperatures, and the like. The catalyst composition can be optimized for best performance (e.g., greatest NOx reduction), depending on the temperature in that particular zone. For example, the first tier 61 can include the lower loading Ag catalyst described above for reacting the second reductant feedstream 48 at the lower exhaust temperatures (e.g., below about 375° C.) with the NOx to produce nitrogen dioxide. The second tier 62 can include the higher loading Ag catalyst composition configured for reacting the first reductant feedstream 47 at higher exhaust temperatures (e.g., above about 375° C.) with the NOx to produce nitrogen dioxide. In a third tier 63, the DOC bed can be disposed; configured for combusting the heaviest hydrocarbons in the third reductant feedstream 49. The SCR catalyst reactor 22 further includes a mixing zone 66 downstream of and in fluid communication with the first zone 60. The mixing zone 66 is configured to circulate the exhaust stream exiting the multiple tiers of the first zone 60. The mixed exhaust stream is then fed to a third zone 68 of the SCR catalyst reactor. The third zone 68 includes a catalyst bed configured to react any remaining unconverted NOx with the nitrogen-containing hydrocarbons (RONO) as described above for FIG. 2. The catalyst composition in the fourth zone 27 can include the RDC.

Referring to FIGS. 1-5, the amount of reductant that is separated in the separation system and fed to the various zones (and/or tiers) of the SCR catalyst reactor 22 and the exhaust conduit 20 can be controlled using NOx sensors and exhaust temperature sensors that can be placed down stream of the emission treatment system. The NOx sensor can measure the concentration of NOx in the treated exhaust steam exiting the system. The NOx sensor can be configured to send a signal representing the NOx concentration in the treated exhaust stream to a reductant flow controller. The reductant flow controller can integrate the processed information and determine if the system parameters are indicative of proper control of the treated exhaust stream, and may further determine whether there is a need for supply of reductants to the various zones of the SCR catalyst reactor 22. Accordingly, the reductant flow controller can regulate the flow of the converted hydrocarbon reductant stream 41 entering the separation system 16, the fuel entering the fuel converter 14, and the reductant feedstreams exiting the separation system 16, based on the signal received from the NOx sensor and/or the exhaust temperature thermocouples. Such a control system can aid in maintaining the optimum utilization of reductant mixture and catalyst bed usage to improve fuel efficiency and maximize emissions reduction in the exhaust stream.

The hydrocarbon reductants converted from the fuel can be used to reduce NOx in the various zones of the SCR catalyst reactor 22, according to the following overall reaction (1).

NOₓ+O₂+organic reductant→N₂+CO₂+H₂O (1)

The exhaust stream usually includes air, water, CO, CO₂, NOₓ, SOₓ, H₂O, and may also include other impurities. Water contained in the exhaust stream is generally in the form of steam. Additionally, uncombusted or incompletely combusted fuel may also be present in the exhaust stream. The hydrocarbon reductant molecules are fed into the exhaust stream to form a gas mixture, which is then fed through the selective catalytic reduction catalyst. Sufficient oxygen to support the NOₓ reduction reaction may already be present in the exhaust stream. If the oxygen present in the exhaust stream is not sufficient for the NOₓ reduction reaction, additional oxygen gas may also be introduced into the exhaust stream in the form of air. In some embodiments, the gas mixture includes from about 1 mole percent (mole %) to about 21 mole % of oxygen gas. In some other embodiments, the gas mixture includes from about 1 mole % to about 15 mole % of oxygen gas. To reiterate, the hydrocarbon reductants are particularly effective for reducing NOx emissions in the exhaust stream, but are more efficient at reduction when utilized at the optimal temperatures over the optimal catalyst bed compositions.

In all of the various exemplary embodiments, the NOx reducing system described above allows for a flexible SCR catalytic process using multiple optimized catalytic beds to provide maximum utilization of a broad diversity of hydrocarbon reductants. The systems described herein combine the proper set of catalyst compositions in the proper order, and inject the proper portion of hydrocarbon-based reductants at the proper locations within the SCR catalyst reactor. Moreover, the system can be easily installed on-board mobile applications and does not require additional chemical storage on-board (such as ammonia or urea found in other NOx treatment systems). This system simply utilizes the fuel required by the engine for power. To reiterate, this system provides flexible solution for optimum utilization of variable-composition reductant streams of hydrogen, carbon monoxide, and C1 and above hydrocarbons for NOx hydrocarbon-based SCR as produced on-board mobile applications.

The terminology used herein is for the purpose of describing particular embodiments only and is not intended to be limiting of the invention. Ranges disclosed herein are inclusive and combinable (e.g., ranges of “up to about 25 wt %, or, more specifically, about 5 wt % to about 20 wt %”, is inclusive of the endpoints and all intermediate values of the ranges of “about 5 wt % to about 25 wt %,” etc.). “Combination” is inclusive of blends, mixtures, alloys, reaction products, and the like. Furthermore, the terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item. The modifier “about” used in connection with a quantity is inclusive of the stated value and has the meaning dictated by context, (e.g., includes the degree of error associated with measurement of the particular quantity). The suffix “(s)” as used herein is intended to include both the singular and the plural of the term that it modifies, thereby including one or more of that term (e.g., the colorant(s) includes one or more colorants). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

Unless otherwise defined, all terms (including technical and scientific terms) used herein have the same meaning as commonly understood by one of ordinary skill in the art to which the embodiments of the invention belong. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

While the disclosure has been described with reference to an exemplary embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

1. A system, comprising:
a fuel converter comprising a catalyst composition, and the catalyst composition can convert fuel into a hydrocarbon reductant stream and/or a syngas;
a separation system that separates the hydrocarbon reductant stream into a first reductant sub-stream that comprises short chain hydrocarbon molecules, and a second reductant sub-stream that comprises long chain hydrocarbon molecules;
a selective catalytic reduction catalyst reactor in fluid communication with the fuel converter, and the catalyst reactor has an inner surface that defines a first zone and a second zone, and the first zone is configured to receive the second reductant sub-stream, and the second zone is configured to receive the first reductant sub-stream; and
an exhaust stream that flows into the first zone contacts the second reductant sub-stream before flowing into the second zone and contacting the first reductant sub-stream.

2. The system of claim 1, wherein the fuel converter performs a selected one or both of a autothermal cracking and a catalytic partial oxidation of the fuel to form the hydrocarbon reductant stream and/or the syngas.

3. The system of claim 2, wherein the catalyst composition is bifunctional and comprises catalytic partial oxidation sites and autothermal cracking sites.

4. The system of claim 3, wherein the catalytic partial oxidation sites comprise platinum, palladium, rhodium, iridium, osmium, ruthenium, or a combination comprising at least one of the foregoing.

5. The system of claim 3, wherein the autothermal cracking sites comprise a zeolite.

6. The system of claim 2, wherein the selective catalytic reduction catalyst reactor receives the hydrogen-rich syngas.

7. The system of claim 1, wherein the separation system comprises two or more separators, wherein one separates the hydrocarbon reductant stream into the first reductant sub-stream, and the second separator separates the hydrocarbon reductant stream into the second reductant sub-stream.

8. The system of claim 7, wherein the first zone comprises a deep oxidation catalyst, wherein the deep oxidation catalyst can combust the second reductant sub-stream.

9. The system of claim 7, wherein the second zone comprises a catalyst composition, wherein the catalyst composition can react the short chain hydrocarbon molecules with one or more components in the exhaust stream.

10. A method, comprising:
converting a fuel into a hydrocarbon reductant stream;
separating the hydrocarbon reductant stream into a plurality of sub-streams, and each of the plurality of streams has a hydrocarbon reductant with a differing average carbon chain length;
feeding the plurality of streams to a selective catalytic reduction catalyst reactor, wherein each of the plurality of sub-streams is fed to a corresponding zone in the reactor so as to contact one of a set of catalyst compositions, and each catalyst composition in the set being configured to function in a determined manner with the carbon chain length of the hydrocarbon reductant of that sub-stream; and
contacting an exhaust stream with the selective catalytic reduction catalyst reactor and the plurality of hydrocarbon reductant sub-streams to control a concentration of one or more components of the exhaust stream.

11. The method of claim 10, further comprising converting the fuel into a hydrogen-rich syngas and a hydrocarbon reductant stream.

12. The method of claim 11, further comprising feeding the hydrogen-rich syngas to the selective catalytic reduction catalyst reactor.

13. The method of claim 10, wherein a first stream of the plurality of sub-streams comprises a plurality of organic molecules having greater than 10 carbon molecules.

14. The method of claim 13, further comprising feeding the first stream to a first zone of the selective catalytic reduction catalyst reactor, wherein the first zone comprises a deep oxidation catalyst, wherein the deep oxidation catalyst can combust the first stream.

15. The method of claim 13, wherein a second stream of the plurality of sub-streams comprises a plurality of organic molecules having less than or equal to 10 carbon molecules.

16. The method of claim 15, further comprising feeding the second stream to each of a second, third, and fourth zone of the selective catalytic reduction catalyst reactor, wherein the second zone comprises a catalyst composition that can react a plurality of organic molecules having 5 to 10 carbon molecules with one or more components in the exhaust stream, wherein the third zone comprises a catalyst composition that can react a plurality of organic molecules having 1 to 4 carbon molecules with one or more components in the exhaust stream, and the fourth zone comprises a catalyst composition that can react any of the remaining plurality of organic molecules with a selected one or all of the reactant products produced in the second and third zones.

17. The method of claim 10, wherein converting the fuel comprises a selected one or both of autothermal cracking and partial oxidation catalysis.

18. The method of claim 16, wherein the catalyst composition of the second zone comprises, based on the total weight of the catalyst composition, about 0.5 percent by weight to about 10 percent by weight silver.

19. The method of claim 10, further comprising atomizing the fuel before converting the fuel.
